(19) United States
(12) Patent Application Publication
(10) Pub. No.: US 2012/0130051 A1
(43) Pub. Date: May 24, 2012

(54) ASTRINGENCY IN SOY PROTEIN SOLUTIONS

(76) Inventors: Sarah Medina, Winnipeg (CA); Kevin I. Segall, Winnipeg (CA)

(21) Appl. No.: 13/304,354

(22) Filed: Nov. 24, 2011

Related U.S. Application Data
(60) Provisional application No. 61/344,946, filed on Nov. 24, 2010.

(51) Int. Cl. C07K 1/14; C07K 14/415; C07K 1/34

(52) U.S. Cl. 530/378

(57) ABSTRACT

A soy protein product having reduced astringency, which may be an isolate, produces transparent heat-stable solutions at low pH values and is useful for the fortification of soft drinks and sports drinks without precipitation of protein. The soy protein product is obtained by extracting a soy protein source material with an aqueous calcium salt solution to form an aqueous soy protein solution, separating the aqueous soy protein solution from residual soy protein source, adjusting the pH of the aqueous soy protein solution to a pH of about 1.5 to about 4.4 using at least one organic acid, such as citric acid or a blend of citric acid and malic acid or a mixture of at least one organic acid and at least one mineral acid, such as hydrochloric acid and phosphoric acid, to produce an acidified clear soy protein solution, which may be dried directly or following optional concentration and diafiltration, to provide the soy protein product.

### REFERENCE TO RELATED APPLICATION

This application claims priority under 35 USC 119(e) from U.S. Provisional Patent Application No. 61/344,946 filed Nov. 24, 2010.

### FIELD OF INVENTION

The present invention relates to the production of protein solutions of reduced astringency from soy.

### BACKGROUND TO THE INVENTION

In copending U.S. patent application Ser. No. 12/603,087 filed Oct. 21, 2009 (US Patent Publication No. 2010-0098818 published Apr. 22, 2010 (S701)) and Ser. No. 12/923,897 filed Oct. 13, 2010 (U.S. Patent Publication No. 2011-0038993 published Feb. 17, 2011 “S701” CIP), assigned to the assignee hereof and the disclosure of which are incorporated herein by reference, there is described the provision of a novel soy protein product having a protein content of at least about 60 wt % (N×6.25) on a dry weight basis, preferably a soy protein isolate having a protein content of at least about 90 wt % (N×6.25) d.b. The soy protein product has a unique combination of properties, namely:
completely soluble in aqueous media at acid pH values of less than about 4.4
heat stable in aqueous media at acid pH values of less than about 4.4
does not require stabilizers or other additives to maintain the protein product in solution
is low in phytic acid
requires no enzymes in the production thereof

In addition, the soy protein product has no beany flavour or off odours characteristic of soy protein products.

This novel soy protein product is prepared by a method which comprises:
(a) extracting a soy protein source with an aqueous calcium chloride solution to cause solubilization of soy protein from the protein source and to form an aqueous soy protein solution,
(b) at least partially separating the aqueous soy protein solution from residual soy protein source,
(c) optionally diluting the aqueous soy protein solution,
(d) adjusting the pH of the aqueous soy protein solution to a pH of about 1.5 to about 4.4, preferably about 2 to about 4, to produce an acidified clear soy protein solution,
(e) optionally polishing the acidified clear soy protein solution to remove residual particulates,
(f) optionally concentrating the aqueous clear soy protein solution while maintaining the ionic strength substantially constant by using a selective membrane technique,
(g) optionally diafiltering the concentrated soy protein solution, and
(h) optionally drying the concentrated soy protein solution.

Under certain conditions aqueous acidic solutions of the novel soy protein product exhibit an astringency which may impair the use of the soy protein product in certain applications.

### SUMMARY OF THE INVENTION

One step of the procedure described in the aforementioned U.S. patent applications Ser. Nos. 12/603,087 and 12/923,897 involves adjustment of the pH of the optionally diluted soy protein solution to a value of about 1.5 to about 4.4, preferably about 2 to about 4, by the addition of any suitable food grade acid to result in a clear acidified aqueous soy protein solution. The only acids specifically identified in applications Ser. Nos. 12/603,087 and 12/923,897 as suitable for the acidifying step are mineral acids.

It has been found that the astringent sensation of aqueous acidic solutions of the novel soy protein product described in applications Ser. Nos. 12/603,087 and 12/923,897 can be significantly reduced by the utilization of an organic acid, such as citric acid or malic acid, for the acidification step. Preferably, citric acid or a blend of citric acid and malic acid is employed. In addition, the astringent sensation is also reduced when citric acid or a citric acid/malic acid blend is employed in combination with an inorganic acid, such as hydrochloric acid or phosphoric acid, in any proportion.

In accordance with one aspect of the present invention, there is provided a method of forming a soy protein product, which comprises:
(a) extracting a soy protein source with an aqueous calcium salt solution, preferably an aqueous calcium chloride solution, to cause solubilization of soy protein from the protein source and to form an aqueous soy protein solution,
(b) at least partially separating the aqueous soy protein solution from residual soy protein source,
(c) optionally diluting the aqueous soy protein solution,
(d) adjusting the pH of the aqueous soy protein solution to a pH of about 1.5 to about 4.4, preferably about 2 to about 4, using at least one organic acid alone or in admixture with at least one mineral acid, preferably citric acid or a blend of citric acid and malic acid, optionally blended with at least one of hydrochloric acid and phosphoric acid, to produce an acidified clear soy protein solution,
(e) optionally polishing the acidified clear soy protein solution to remove residual particulates,
(f) optionally concentrating the aqueous clear soy protein solution while maintaining the ionic strength substantially constant by using a selective membrane technique,
(g) optionally diafiltering the concentrated soy protein solution, and
(h) optionally drying the concentrated and optionally diafiltered soy protein solution.

The soy protein product produced according to the process herein lacks the characteristic beany flavour of soy protein products and is suitable, not only for protein fortification of acid media such as soft drinks and sports drinks, but may be used in a wide variety of conventional applications of protein products, including but not limited to protein fortification of processed foods and beverages, emulsification of oils, as a body former in baked goods and foaming agent in products which entrap gases. In addition, the soy protein product may be formed into protein fibers, useful in meat analogs and may be used as an egg white substitute or extender in food products where egg white is used as a binder. The soy protein product may also be used in nutritional supplements. The soy protein product may also be used in dairy analog products or products which are dairy/soy blends. Other uses of the soy protein product are in pet foods, animal feed and in industrial and cosmetic applications and in personal care products.

### General Description of Invention

The initial step of the process of providing the soy protein product involves solubilizing soy protein from a soy protein source. The soy protein source may be soybeans or any soy product or byproduct derived from the processing of soybeans, including but not limited to soy meal, soy flakes, soy grits and soy flour. The soy protein source may be used in the full fat form, partially defatted form or fully defatted form. Where the soy protein source contains an appreciable amount of fat, an oil-removal step generally is required during the process. The soy protein recovered from the soy protein source may be the protein naturally occurring in soybean or the proteinaceous material may be a protein modified by genetic manipulation but possessing characteristic hydrophobic and polar properties of the natural protein.

Protein solubilization from the soy protein source material is effected most conveniently using calcium chloride solution, although solutions of other calcium salts may be used. In addition, other alkaline earth metal compounds may be used, such as magnesium salts. Further, extraction of the soy protein from the soy protein source may be effected using calcium salt solution in combination with another salt solution, such as sodium chloride. Additionally, extraction of the soy protein from the soy protein source may be effected using water or other salt solution, such as sodium chloride, with calcium salt subsequently being added to the aqueous soy protein solution produced in the extraction step. Precipitate formed upon addition of the calcium salt is removed prior to subsequent processing.

As the concentration of the calcium salt solution increases, the degree of solubilization of protein from the soy protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In a batch process, the salt solubilization of the protein is effected at a temperature of from about 1° C. to about 100° C., preferably about 15° to about 65° C., more preferably about 50° C. to about 60° C., preferably accompanied by agitation to decrease the solubilization time, which is usually about 1 to about 60 minutes. It is preferred to effect the solubilization to extract substantially as much protein from the soy protein source as is practicable, so as to provide an overall high product yield.

In a continuous process, the extraction of the soy protein from the soy protein source is carried out in any manner consistent with effecting a continuous extraction of soy protein from the soy protein source. In one embodiment, the soy protein source is continuously mixed with the calcium salt solution and the mixture is conveyed through a pipe or conduit having a length and at a flow rate for a residence time sufficient to effect the desired extraction in accordance with the parameters described herein. In such a continuous procedure, the salt solubilization step is effected rapidly, in a time of up to about 10 minutes, preferably to effect solubilization to extract substantially as much protein from the soy protein source as is practicable. The solubilization in the continuous procedure is effected at temperatures between about 1° C. and about 100° C., preferably about 15° to about 65° C., more preferably between about 50° C. and about 60° C.

The extraction is generally conducted at a pH of about 5 to about 11, preferably about 5 to about 7. The pH of the extraction system (soy protein source and calcium salt solution) may be adjusted to any desired value within the range of about 5 to about 11 for use in the extraction step by the use of any convenient food grade acid or food grade alkali as required.

The concentration of soy protein source in the calcium salt solution during the solubilization step may vary widely. Typical concentration values are about 5 to about 15% w/v.

The protein extraction step with the aqueous salt solution has the additional effect of solubilizing fats which may be present in the soy protein source, which then results in the fats being present in the aqueous phase.

The protein solution resulting from the extraction step generally has a protein concentration of about 5 to about 50 g/L, preferably about 10 to about 50 g/L.

The aqueous calcium salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous phase resulting from the extraction step then may be separated from the residual soy protein source, in any convenient manner, such as by employing a decanter centrifuge or any suitable sieve, followed by disc centrifugation and/or filtration, to remove residual soy protein source material. The separated residual soy protein source may be dried for disposal. Alternatively, the separated residual soy protein source may be processed to recover some residual protein. The separated residual soy protein source may be re-extracted with fresh calcium salt solution and the protein solution yielded upon clarification combined with the initial protein solution for further processing as described below. Alternatively, the separated residual soy protein source may be processed by a conventional isoelectric precipitation procedure or any other convenient procedure to recover residual protein.

Where the soy protein source contains significant quantities of fat, as described in U.S. Pat. Nos. 5,844,086 and 6,005,076, assigned to the assignee hereof and the disclosures of which are incorporated herein by reference, then the defatting steps described therein may be effected on the separated aqueous protein solution. Alternatively, defatting of the separated aqueous protein solution may be achieved by any other convenient procedure.

The aqueous soy protein solution may be treated with an adsorbent, such as powdered activated carbon or granulated activated carbon, to remove colour and/or odour compounds. Such adsorbent treatment may be carried out under any convenient conditions, generally at the ambient temperature of the separated aqueous protein solution. For powdered activated carbon, an amount of about 0.025% to about 5% w/v, preferably about 0.05% to about 2% w/v, is employed. The adsorbing agent may be removed from the soy solution by any convenient means, such as by filtration.

The resulting aqueous soy protein solution may be diluted generally with about 0.5 to about 10 volumes, preferably about 0.5 to about 2 volumes, of aqueous diluent in order to decrease the conductivity of the aqueous soy protein solution to a value of generally below about 90 mS, preferably about 4 to about 18 mS. Such dilution is usually effected using water, although dilute salt solution, such as sodium chloride or calcium chloride, having a conductivity of up to about 3 mS, may be used.

The diluent with which the soy protein solution is mixed may have a temperature of about 1° to about 100° C., preferably about 15° to about 65° C., more preferably about 50° to about 60° C.

The optionally diluted soy protein solution then is adjusted in pH to a value of about 1.5 to about 4.4, preferably about 2 to about 4, by the addition of at least one organic acid, to result in a clear acidified aqueous soy protein solution. The clear acidified soy protein solution has a conductivity of generally below about 95 mS for a diluted soy protein solution or generally below about 115 mS for an undiluted soy protein solution, in both cases preferably about 4 to about 23 mS, The organic acid utilized in the acidifying step is preferably citric acid or a blend of citric acid and malic acid which may be used in combination with an inorganic acid, such as hydrochloric acid or phosphoric acid, in any proportion.

The clear acidified aqueous soy protein solution may be subjected to a heat treatment to inactivate heat labile anti-nutritional factors, such as trypsin inhibitors, present in such solution as a result of extraction from the soy protein source material during the extraction step. Such a heating step also provides the additional benefit of reducing the microbial load. Generally, the protein solution is heated to a temperature of about 70° to about 160° C., for about 10 seconds to about 60 minutes, preferably about 80° to about 120° C. for about 10 seconds to about 5 minutes, more preferably about 85° to about 95° C., for about 30 seconds to about 5 minutes. The heat treated acidified soy protein solution then may be cooled for further processing as described below, to a temperature of about 2° to about 65° C., preferably about 50° C. to about 60° C.

The optionally diluted, acidified and optionally heat treated protein solution may optionally be polished by any convenient means, such as by filtering, to remove any residual particulates.

The resulting clear acidified aqueous soy protein solution may be directly dried to produce a soy protein product. In order to provide a soy protein product having a decreased impurities content and a reduced salt content, such as a soy protein isolate, the clear acidified aqueous soy protein solution may be processed prior to drying.

The clear acidified aqueous soy protein solution may be concentrated to increase the protein concentration thereof while maintaining the ionic strength thereof substantially constant. Such concentration generally is effected to provide a concentrated soy protein solution having a protein concentration of about 50 to about 300 g/L, preferably about 100 to about 200 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 1,000,000 Daltons, preferably about 5,000 to about 100,000 Daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include not only the ionic species of the food grade salt but also low molecular weight materials extracted from the source material, such as carbohydrates, pigments, low molecular weight proteins and anti-nutritional factors, such as trypsin inhibitors, which are themselves low molecular weight proteins. The molecular weight cut-off of the membrane is usually chosen to ensure retention of a significant proportion of the protein in the solution, while permitting contaminants to pass through having regard to the different membrane materials and configurations.

The concentrated soy protein solution then may be subjected to a diafiltration step using water or a dilute saline solution. The diafiltration solution may be at its natural pH or at a pH equal to that of the protein solution being diafiltered or at any pH value in between. Such diafiltration may be effected using from about 1 to about 40 volumes of diafiltration solution, preferably about 2 to about 25 volumes of diafiltration solution. In the diafiltration operation, further quantities of contaminants are removed from the clear aqueous soy protein solution by passage through the membrane with the permeate. This purifies the clear aqueous protein solution and may also reduce its viscosity. The diafiltration operation may be effected until no significant further quantities of contaminants or visible colour are present in the permeate or until the retentate has been sufficiently purified so as, when dried, to provide a soy protein isolate with a protein content of at least about 90 wt % (N×6/5) d.b. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 1,000,000 Daltons, preferably about 5,000 to about 100,000 Daltons, having regard to different membrane materials and configuration.

Alternatively, the diafiltration step may be applied to the clear acidified aqueous protein solution prior to concentration or to the partially concentrated clear acidified aqueous protein solution. Diafiltration may also be applied at multiple points during the concentration process. When diafiltration is applied prior to concentration or to the partially concentrated solution, the resulting diafiltered solution may then be additionally concentrated. The viscosity reduction achieved by diafiltering multiple times as the protein solution is concentrated may allow a higher final, fully concentrated protein concentration to be achieved. This reduces the volume of material to be dried.

The concentration step and the diafiltration step may be effected herein in such a manner that the soy protein product subsequently recovered contains less than about 90 wt % protein (N×6.25) d.b., such as at least about 60 wt % protein (N×6.25) d.b. By partially concentrating and/or partially diafiltering the clear aqueous soy protein solution, it is possible to only partially remove contaminants. This protein solution may then be dried to provide a soy protein product with lower levels of purity. The soy protein product is still able to produce clear protein solutions under acidic conditions.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the concentrated soy protein solution.

The concentration step and the optional diafiltration step may be effected at any convenient temperature, generally about 2° to about 65° C., preferably about 50° to about 60° C., and for the period of time to effect the desired degree of concentration and diafiltration. The temperature and other conditions used to some degree depend upon the membrane equipment used to effect the membrane processing, the desired protein concentration of the solution and the efficiency of the removal of contaminants to the permeate.

There are two main trypsin inhibitors in soy, namely the Kunitz inhibitor, which is a heat-labile molecule with a molecular weight of approximately 21,000 Daltons, and the Bowman-Birk inhibitor, a more heat-stable molecule with a molecular weight of about 8,000 Daltons. The level of trypsin inhibitor activity in the final soy protein product can be controlled by manipulation of various process variables.

As noted above, heat treatment of the clear acidified aqueous soy protein solution may be used to inactivate heat-labile trypsin inhibitors. The partially concentrated or filly concentrated acidified soy protein solution may also be heat treated to inactivate heat labile trypsin inhibitors. When the heat treatment is applied to the partially concentrated acidified soy protein solution, the resulting heat treated solution may then be additionally concentrated.

In addition, the concentration and/or diafiltration steps may be operated in a manner favorable for removal of trypsin inhibitors in the permeate along with the other contaminants. Removal of the trypsin inhibitors is promoted by using a membrane of larger pore size, such as about 30,000 to about 1,000,000 Da, operating the membrane at elevated temperatures, such as about 30° to about 65° C., preferably about 50° to about 60° C., and employing greater volumes of diafiltration medium, such as about 10 to about 40 volumes.

Acidifying and membrane processing the diluted protein solution at a lower pH of about 1.5 to about 3 may reduce the trypsin inhibitor activity relative to processing the solution at higher pH of about 3 to about 4.4. When the protein solution is concentrated and diafiltered at the low end of the pH range, it may be desired to raise the pH of the retentate prior to drying. The pH of the concentrated and diafiltered protein solution may be raised to the desired value, for example pH 3, by the addition of any convenient food grade alkali such as sodium hydroxide.

Further, a reduction in trypsin inhibitor activity may be achieved by exposing soy materials to reducing agents that disrupt or rearrange the disulfide bonds of the inhibitors. Suitable reducing agents include sodium sulfite, cysteine and N-acetylcysteine.

The addition of such reducing agents may be effected at various stages of the overall process. The reducing agent may be added with the soy protein source material in the extraction step, may be added to the clarified aqueous soy protein solution following removal of residual soy protein source material, may be added to the concentrated protein solution before or after diafiltration or may be dry blended with the dried soy protein product. The addition of the reducing agent may be combined with a heat treatment step and the membrane processing steps, as described above.

If it is desired to retain active trypsin inhibitors in the concentrated protein solution, this can be achieved by eliminating or reducing the intensity of the heat treatment step, not utilizing reducing agents, operating the concentration and diafiltration steps at the higher end of the pH range, such as pH 3 to about 4.4, utilizing a concentration and diafiltration membrane with a smaller pore size, operating the membrane at lower temperatures and employing fewer volumes of diafiltration medium.

The concentrated and optionally diafiltered protein solution may be subject to a further defatting operation, if required, as described in U.S. Pat. Nos. 5,844,086 and 6,005,076. Alternatively, defatting of the concentrated and optionally diafiltered protein solution may be achieved by any other convenient procedure.

The concentrated and optionally diafiltered clear aqueous protein solution may be treated with an adsorbent, such as powdered activated carbon or granulated activated carbon, to remove colour and/or odour compounds. Such adsorbent treatment may be carried out under any convenient conditions, generally at the ambient temperature of the concentrated protein solution. For powdered activated carbon, an amount of about 0.025% to about 5% w/v, preferably about 0.05% to about 2% w/v, is employed. The adsorbent may be removed from the soy protein solution by any convenient means, such as by filtration.

The concentrated and optionally diafiltered clear aqueous soy protein solution may be dried by any convenient technique, such as spray drying or freeze drying. A pasteurization step may be effected on the soy protein solution prior to drying. Such pasteurization may be effected under any desired pasteurization conditions. Generally, the concentrated and optionally diafiltered soy protein solution is heated to a temperature of about 55° to about 70° C., preferably about 60° to about 65° C., for about 30 seconds to about 60 minutes, preferably about 10 minutes to about 15 minutes. The pasteurized concentrated soy protein solution then may be cooled for drying, preferably to a temperature of about 25° to about 40° C.

The dry soy protein product has a protein content in excess of about 60 wt %, (N×6.25) d.b. Preferably, the dry soy protein product is an isolate with a high protein content, in excess of about 90 wt % protein, preferably at least about 100 wt % (N×625) d.b.

The soy protein product produced herein is soluble in an acidic aqueous environment, making the product ideal for incorporation into beverages, both carbonated and uncarbonated, to provide protein fortification thereto. Such beverages have a wide range of acidic pH values, ranging from about 2.5 to about 5. The soy protein product provided herein may be added to such beverages in any convenient quantity to provide protein fortification to such beverages, for example, at least about 5 g of the soy protein per serving. The added soy protein product dissolves in the beverage and does not impair the clarity of the beverage, even after thermal processing. The soy protein product may be blended with dried beverage prior to reconstitution of the beverage by dissolution in water. In some cases, modification to the normal formulation of the beverages to tolerate the composition of the invention may be necessary where components present in the beverage may adversely affect the ability of the composition of the invention to remain dissolved in the beverage.

### EXAMPLES

### Example 1

This Example compares the astringency of a soy protein product prepared using acidification with organic acid with the astringency of a soy protein product prepared using acidification with HCl. The astringency of the protein products was compared by sensory evaluation in a commercial beverage.

30 kg of defatted soy white flake was added to 300 L of 015 M CaCl₂ solution at ambient temperature and agitated for 30 minutes to provide an aqueous protein solution. The residual soy white flake was removed and the resulting protein solution was clarified by centrifugation to provide ‘a’ L of protein solution having a protein content of ‘b’ % by weight.

‘c’ L of protein solution was then added to L of reverse osmosis purified water and the pH of the sample lowered to ‘e’ with a solution of ‘f’. The diluted and acidified solution was heat treated at 90° C. for 30 seconds ‘g’.

The heat treated acidified protein solution was reduced in volume from ‘h’ L to ‘i’ L by concentration on a polyethersulfone membrane, having a molecular weight cutoff of 100,000 Daltons, operated at a temperature of approximately ‘j’ ° C. At this point, the acidified protein solution, with a protein content of ‘k’ wt %, was diafiltered with L of reverse osmosis (RO) purified water, with the diafiltration operation conducted at approximately ‘m’ ° C. The diafiltered solution was then further concentrated to a volume of L and diafiltered with an additional ‘o’ L of RO water, with the diafiltration operation conducted at approximately ‘p’ ° C. The protein solution before spray drying was recovered in a yield of ‘q’ wt % of the initial centrifuged protein solution. The acidified, diafiltered, concentrated protein solution was then dried to yield a product found to have a protein content of ‘r’ % (N×6.25) d.b. The product was given designation ‘s’ S701H. The parameters ‘a’ to ‘s’ for two runs are set forth in the following Table 1.

TABLE 1
Parameters for the production of S701H

| s | S016-K02-09A | S016-K03-09A |
|---|---|---|
| a | 264 | 244.3 |
| b | 2.72 | 2.44 |
| c | 264 | 244.3 |
| d | 196 | 245 |
| e | 2.69 | 2.89 |
| f | HCl | Citric acid |
| g | and then filtered | |
| h | 460 | 513 |
| i | 106 | 101 |
| j | 30 | 30 |
| k | 5.06 | 4.90 |
| l | 138 | 125 |
| m | 31 | 30 |
| n | 53 | 50 |
| o | 405 | 375 |
| p | 30 | 30 |
| q | 65.7 | 79.2 |
| r | 100.96 | 100.64 |

An informal taste panel was presented with blind samples of 8016-K 02-09A S701H and S016-1(03-09A S701H prepared by dissolving 2 g of protein per 100 ml of the Cherry flavour of the commercial beverage called Kool Aid Jammers. The panelists were asked to identify which sample they felt was more astringent as well as which sample they preferred overall.

Five out of seven panelists felt that the sample containing S016-K02-09A S701H was more astringent than the sample containing S016-K03-09A S701H. Four out of seven panelists preferred the sample containing S016-K03-09A S701H. Comments recorded regarding the sample containing S016-K03-09A S701H included “fruitier”, “nicer flavour”, “less astringent”, “less initial sourness”, “slightly sweeter” and “not as astringent”.

### Example 2

This Example compares the astringency of a blend of soy protein products prepared using acidification with organic acid with the astringency of a blend of soy protein products prepared using acidification with HCl. The astringency of the protein products was compared by sensory evaluation in a commercial beverage.

30 kg of defatted soy white flake was added to 300 L of ‘a’ M CaCl₂ solution at ambient temperature and agitated for 30 minutes to provide an aqueous protein solution. The residual soy white flake was removed and the resulting protein solution was clarified by centrifugation to provide ‘b’ L of protein solution having a protein content of ‘c’ % by weight.

‘d’ L of protein solution was then added to ‘e’ L of reverse osmosis purified water and the pH of the sample lowered to ‘f’ with a solution of ‘g’. The diluted and acidified solution was then heat treated at 90° C. for 30 seconds.

The heat treated acidified protein solution was reduced in volume from ‘h’ L to ‘i’ L by concentration on a polyethersulfone membrane, having a molecular weight cutoff of 100,000 Daltons, operated at a temperature of approximately ‘j’ ° C. At this point, the acidified protein solution, with a protein content of ‘k’ wt %, was diafiltered with ‘l’ L of reverse osmosis (RO) purified water, with the diafiltration operation conducted at approximately ‘m’ ° C. The diafiltered solution was then further concentrated to a volume of ‘n’ L and diafiltered with an additional ‘o’ L of RO water, with the diafiltration operation conducted at approximately ‘p’ ° C. After this second diafiltration, the protein solution was concentrated from a protein content of ‘q’ to a protein content of ‘r’ % by weight then diluted to a protein content of ‘s’ % by weight with water to facilitate spray drying. The protein solution before spray drying was recovered in a yield of ‘t’ wt % of the initial centrifuged protein solution. The acidified, diafiltered, concentrated and diluted protein solution was then dried to yield a product found to have a protein content of ‘u’% (N×6.25) d.b. The product was given designation ‘v’ S701H. The parameters ‘a’ to ‘v’ for seven runs are set forth in the following Table 2.

TABLE 2
Parameters for the production of S701H

| | S019- | S019- | S019- | S019- | S019- | S019- | S019- |
|---|---|---|---|---|---|---|---|
| v | F21-10A | F22-10A | D15-10A | D19-10A | D20-10A | D21-10A | D26-10A |
| a | 0.13 | 0.13 | 0.15 | 0.15 | 0.15 | 0.15 | 0.15 |
| b | 232 | 255.3 | 209.3 | 233 | 228 | 221 | 240 |
| c | 2.50 | 2.46 | 2.76 | 2.83 | 2.69 | 2.79 | 2.57 |
| d | 232 | 255.3 | 209.3 | 233 | 228 | 221 | 240 |
| e | 165 | 155.4 | 220 | 245 | 249 | 239 | 240 |
| f | 3.14 | 3.11 | 3.27 | 3.14 | 3.05 | 3.29 | 3.00 |
| g | malic acid | citric acid | HCl | HCl | HCl | HCl | HCl |
| h | 400 | 395 | 408 | 485 | 500 | 480 | 505 |
| i | 102 | 95 | 89 | 96 | 108 | 107 | 112 |
| j | 41 | 41 | 30 | 50 | 29 | 50 | 30 |
| k | 4.75 | 4.64 | 4.99 | 5.81 | 4.77 | 4.73 | 4.65 |
| l | 153 | 142 | 134 | 144 | 162 | 160 | 168 |
| m | 40 | 41 | 30 | 51 | 29 | 50 | 29 |
| n | 40 | 40 | 41 | 48 | 47 | 48 | 48 |
| o | 300 | 300 | 308 | 360 | 353 | 360 | 360 |
| p | 41 | 41 | 30 | 49 | 30 | 51 | 30 |
| q | 10.15 | 10.02 | 9.66 | 10.85 | 9.89 | 9.34 | 9.80 |
| r | 12.12 | 12.04 | 11.78 | 13.49 | 11.78 | 11.86 | 12.11 |
| s | 6.27 | 6.06 | 5.94 | 6.22 | 5.02 | 5.55 | 6.00 |
| t | 68.8 | 63.5 | 74.0 | 79.2 | 66.4 | 77.3 | 78.3 |
| u | 101.77 | 101.39 | 100.53 | 102.43 | 102.10 | 102.45 | 102.22 |

Batches of S701H were dry blended in the proportions shown below to provide a composite product called Organic acid blend A S701H (Table 3) and Clarisoy XIII S701H (Table 4). Organic acid blend A S701H was formulated in such a way that half of the protein content came from batch S019-F21-10A S701H and half from S019-F22-10A S701H,

TABLE 3
Proportion of products in Organic acid blend A S701H

| | Proportion of |
|---|---|
| | total product |
| Batch | weight (%) |
| S019-F21-10A | 50.2 |
| S019-F22-10A | 49.8 |

TABLE 4
Proportion of products in Clarisoy XIII S701H

| | Proportion of |
|---|---|
| | total product |
| Batch | weight (%) |
| S019-D15-10A | 16.9 |
| S019-D19-10A | 21.7 |
| S019-D20-10A | 21.2 |
| S019-D21-10A | 20.7 |
| S019-D26-10A | 19.5 |

An informal taste panel was presented with blind samples of Organic acid blend A S701H and Clarisoy XIII S701H prepared by dissolving 2 g protein per 100 ml of the Cherry flavour of the commercial beverage called Kool Aid Jammers. The panelists were asked to identify which sample they felt was more astringent as well as which sample they preferred overall.

Six out of seven panelists felt that the sample containing Clarisoy XIII S701H was more astringent than the sample containing Organic acid blend A S701H. Six out of seven panelists preferred the sample containing Organic acid blend A S701H. Comments recorded regarding the sample containing Organic acid blend A S701H included “very little astringency at all”, “good cherry taste” and “nice, clean taste”.

### Example 3

This Example compares the astringency of a soy protein product prepared using acidification with a blend of organic acids with the astringency of a blend of soy protein products prepared using acidification with HCl. The astringency of the protein products was compared by sensory evaluation in a commercial beverage.

35 kg of defatted soy white flake was added to 350 L of 0.13 M CaCl₂ solution at ambient temperature and agitated for 30 minutes to provide an aqueous protein solution. The residual soy white flake was removed and the resulting protein solution was clarified by sieving and centrifugation to provide 250 L of protein solution having a protein content of 2.46% by weight.

250 L of protein solution was then added to 193 L of reverse osmosis purified water and the pH of the sample lowered to 3.07 with a solution prepared by dissolving equal weights of malic acid and citric acid in water. The diluted and acidified protein solution was then heat treated at 90° C. for 30 seconds.

The heat treated acidified protein solution was reduced in volume from 440 L to 102 L by concentration on a polyethersulfone membrane, having a molecular weight cutoff of 100,000 Daltons, operated at a temperature of approximately 51° C. At this point, the acidified protein solution, with a protein content of 5.04 wt %, was diafiltered with 163 L of reverse osmosis (RO) purified water, with the diafiltration operation conducted at approximately 50° C. The diafiltered solution was then further concentrated to a volume of 44 L and diafiltered with an additional 330 L of RO water, with the diafiltration operation conducted at approximately 50° C. After this second diafiltration, the protein solution was concentrated from a protein content of 9.79 to a protein content of 12.02% by weight then diluted to a protein content of 5.94% by weight with water to facilitate spray drying. The protein solution before spray drying was recovered in a yield of 73.8 wt % of the initial centrifuged protein solution. The acidified, diafiltered, concentrated and diluted protein solution was then dried to yield a product found to have a protein content of 100.56% (N×6.25) d.b. The product was given designation S020-G13-10A S701H.

An informal taste panel was presented with blind samples of S020-G13-10A S701H and Clarisoy XIII S701H prepared by dissolving 2 g of protein per 100 ml of the Cherry flavour of the commercial beverage called Kool Aid Jammers. The panelists were asked to identify which sample they felt was more astringent as well as which sample they preferred overall.

Five out of six panelists felt that the sample containing Clarisoy XIII S701H was more astringent than the sample containing S020-G13-10A S701H. Five out of six panelists preferred the sample containing S020-G13-10A S701H. Comments recorded regarding the sample containing S020-G1340A S701H included “sweeter and better cherry taste” and “less astringent”.

### Example 4

This Example is a repeat of Example 3 but utilizing a different flavour of commercial beverage. An informal taste panel was presented with blind samples of S020-G13-10A S701H and Clarisoy XIII S701H prepared by dissolving 2 g protein per 100 m1 of the Strawberry Kiwi flavour of the commercial beverage called Kool Aid Jammers. The panelists were asked to identify which sample they felt was more astringent as well as which sample they preferred overall.

Three out of five panelists felt that the sample containing Clarisoy XIII S701H was more astringent than the sample containing S020-G13-10A S701H. Three out of five panelists preferred the sample containing S020-G13-10A S701H. Comments recorded regarding the sample containing S020-G13-10A S701H included “slightly sweeter”.

### Example 5

This Example compares the same protein samples as Examples 3 and 4, but this time the evaluation was done in purified drinking water rather than a flavoured beverage. An informal taste panel was presented with blind samples of S020-G13-10A S701H and Clarisoy XIII S701H prepared by dissolving 2 g protein per 100 ml of purified drinking water. The panelists were asked to identify which sample they felt was more astringent as well as which sample they preferred overall.

Five out of seven panelists felt that the sample containing Clarisoy XIII S701H was more astringent than the sample containing S020-G1340A S701H. Five out of seven panelists preferred the sample containing S020-G13-10A S701H. Comments recorded regarding the sample containing S020-G13-10A S701H included “bland” and “much less astringent”.

### Example 6

This Example compares the astringency of a blend of soy protein products prepared using acidification with organic acid and soy protein product prepared using acidification with HCl with the astringency of a blend of protein product prepared using acidification with HCl alone. The astringency of the protein products was compared by sensory evaluation in purified drinking water.

Batches of S701H were dry blended in the proportions shown below to provide a composite product called Organic acid/HCl blend A S701H (Table 5). Organic acid/HCl blend A S701H was formulated in such as a way that half of the protein content came from batch S020-G13-10A S701H and half from Clarisoy XIII S701H.

TABLE 5
Proportion of products in Organic acid/HCl blend A S701H

| | Proportion of |
|---|---|
| | total product |
| Batch | weight (%) |
| S020-G13-10A | 49.8 |
| Clarisoy XIII | 50.2 |

An informal taste panel was presented with blind samples of Organic acid/HCl blend A S701H and Clarisoy XIII S701H prepared by dissolving 2 g protein per 100 ml of purified drinking water. The panelists were asked to identify which sample they felt was more astringent as well as which sample they preferred overall.

Five out of seven panelists felt that the sample containing Clarisoy XIII S701H was more astringent than the sample containing the Organic acid/HCl blend A S701H. Five out of seven panelists preferred the sample containing the Organic acid/HCl blend A S701H. Comments recorded regarding the sample containing the Organic acid/HCl blend A S701H included “less astringent”, “sweeter and better overall taste”, “clean flavour” and “almost no astringency”.

### SUMMARY OF THE DISCLOSURE

In summary of this disclosure, a soy protein product, which may be an isolate, produces transparent heat-stable solutions of reduced astringency at low pH values and is useful for the fortification of soft drinks and sports drinks without the precipitation of protein. The soy protein product is obtained by extracting a soy protein source material with an aqueous calcium salt solution to form an aqueous soy protein solution, separating the aqueous soy protein solution from residual soy protein source, adjusting the pH of the aqueous soy protein solution to a pH of about 1,5 to about 4.4 using at least one organic acid to produce an acidified clear protein solution, which may be dried, following optional concentration and diafiltration, to provide the soy protein product. Modifications are possible within the scope of the invention.

1. A process for the preparation of a soy protein solution, which comprises:
(a) extracting a soy protein source with an aqueous calcium salt solution to cause solubilization of soy protein from the soy protein source and to form an aqueous soy protein solution,
(b) at least partially separating the aqueous soy protein solution from residual soy protein source, and
(c) adjusting the pH of the aqueous soy protein solution to a pH of about 1.5 to about 4.4 using at least one organic acid alone or in admixture with at least one mineral acid, to produce an acidified clear soy protein solution.

2. The process of claim 1 wherein said at least one organic acid is citric acid or a blend of citric acid and malic acid.

3. The process of claim 1 wherein said at least one organic acid in admixture with at least one mineral acid is citric acid or a blend of citric acid and malic acid blended with at least one mineral acid.

4. The process of claim 3 wherein said at least one mineral acid is at least one of hydrochloric acid and phosphoric acid.

5. The process of claim 1 wherein said extraction step is effected using an aqueous calcium chloride solution having a concentration of less than about 1.0 M, preferably about 0.10 to about 0.15 M.

6. The process of claim 1 wherein said extraction step is effected at a temperature of about 1° to about 100° C., preferably about 15° to about 65° C., more preferably about 50° to about 60° C.

7. The process of claim 1 wherein said extraction with aqueous calcium salt solution is conducted at a pH of about 5 to about 11, preferably about 5 to about 7.

8. The process of claim 1 wherein said aqueous soy protein solution has a protein concentration of about 5 to about 50 g/L, preferably about 10 to about 50 g/L.

9. The process of claim 1 wherein said aqueous calcium salt solution contains an antioxidant.

10. The process of claim 1 wherein, following said separation step and prior to said pH adjustment step, said aqueous soy protein solution is treated with an adsorbent to remove colour and/or odour compounds from the aqueous soy protein solution.

11. The process of claim 1 wherein, following said separation step and prior to said pH adjustment step, said aqueous soy protein solution is diluted to a conductivity of less than about 90 mS, preferably with about 0.5 to about 10 volumes of aqueous diluent to provide a conductivity of said soy protein solution of about 4 to about 18 mS.

12. The process of claim 11 wherein said aqueous diluent has a temperature of about 1° to about 100° C., preferably about 15° to about 65° C., more preferably about 50° to about 60° C.

13. The process of claim 1 wherein said acidified soy protein solution has a conductivity of less than about 95 mS, preferably about 4 to about 23 mS.

14. The process of claim 1 wherein the pH of said aqueous soy protein solution is adjusted to about pH 2 to about 4.

15. The process of claim 1 wherein the pH-adjusted soy protein solution is subjected to a polishing step.

16. The process of claim 1, wherein said acidified aqueous protein solution is subjected to a heat treatment step to inactivate heat-labile anti-nutritional factors, preferably heat-labile trypsin inhibitors, optionally wherein the heat treatment step also reduces the microbial load in the acidified aqueous protein solution.

17. The process of claim 16, wherein said heat treatment is effected at a temperature of about 70° to about 160° C. for about 10 seconds to about 60 minutes, preferably about 80° to about 120° C. for about 10 seconds to about 5 minutes, more preferably about 85° C. to about 95° C. for about 30 seconds to about 5 minutes.

18. The process of claim 16 wherein the heat treated acidified soy protein solution is cooled to a temperature of about 2° to about 65° C., preferably about 50° to about 60° C., for further processing.

19. The process of claim 16 wherein the heat treated soy protein solution is subjected to a polishing step.

20. The process of claim 1 wherein said acidified clear soy protein solution is dried to provide a soy protein product having a soy protein content of at least about 60 wt % (N×6.25) d.b.

21. The process of claim 1 wherein said acidified clear soy protein solution is concentrated while maintaining the ionic strength thereof substantially constant to produce a concentrated acidified clear soy protein solution having a protein concentration of about 50 to about 300 g/L, preferably about 100 to about 200 g/L, and the concentrated acidified clear soy protein solution is optionally diafiltered.

22. The process of claim 21 wherein said concentration step is effected by ultrafiltration using a membrane having a molecular weight cut-off of about 3,000 to about 1,000,000 Daltons, preferably about 5,000 to about 100.000 Daltons.

23. The process of claim 21 wherein a diafiltration step is effected using water, acidified water, dilute saline or acidified dilute saline on the acidified clear soy protein solution before or after partial or complete concentration thereof.

24. The process of claim 23 wherein said diafiltration is effected using about 1 to about 40 volumes of diafiltration solution, preferably about 2 to about 25 volumes of diafiltration solution.

25. The process of claim 23 wherein said diafiltration is effected until no significant further quantities of contaminants or visible colour are present in the permeate.

26. The process of claim 23 wherein said diafiltration is effected until the retentate has been sufficiently purified so as, when dried, to provide a soy protein isolate with a protein content of at least about 90 wt % (N×6.25) d.b.

27. The process of claim 23 wherein said diafiltration is effected using a membrane having a molecular weight cutoff of about 3,000 to about 1,000,000 Daltons, preferably about 5,000 to about 100,000 Daltons.

28. The process of claim 23 wherein an antioxidant is present in the diafiltration medium during at least part of the diafiltration step.

29. The process of claim 21 wherein said concentration step and optional diafiltration step are carried out at a temperature of about 2° to about 65° C., preferably about 50° to about 60° C.

30. The process of claim 21 wherein the partially concentrated or concentrated and optionally diafiltered acidified clear soy protein solution is subjected to a heat treatment step to inactivate heat-labile anti-nutritional factors, including heat-labile trypsin inhibitors.

31. The process of claim 30 wherein said heat treatment is effected at a temperature of about 70° to about 160° C. for about 10 seconds to about 60 minutes, preferably a temperature of about 80° C. to about 120° C. for about 10 seconds to about 5 minutes, more preferably about 85° C. to about 95° C. for about 30 seconds to about 5 minutes.

32. The process of claim 31 wherein the heat treated soy protein solution is cooled to a temperature of about 2° to about 65° C., preferably about 50° to about 60° C., for further processing.

33. The process of claim 1 wherein said acidified clear soy protein solution is concentrated and/or diafiltered while maintaining the ionic strength thereof substantially constant to produce a concentrated and/or diafiltered acidified clear soy protein solution which, when dried, provides a soy protein product having a protein concentration of at least about 60 wt % (N×6.25) d.b.

34. The process of claim 21 wherein said concentrated and optionally diafiltered acidified clear soy protein solution is treated with an adsorbent to remove colour and/or odour compounds.

35. The process of claim 21 wherein said concentrated and optionally diafiltered acidified clear soy protein solution is pasteurized prior to drying, wherein said pasteurization step preferably is effected at a temperature of about 55° to about 70° C. for about 30 seconds to about 60 minutes, more preferably at a temperature of about 60° to about 65° C. for about 10 to about 15 minutes.

36. The process of claim 26 wherein said concentrated and diafiltered acidified clear soy protein solution is dried to provide a soy protein isolate having a protein content of at least about 90 wt % (N×6.25) d.b, preferably at least about 100 wt % (N×6.25) d.b.

37. The process of claim 21 wherein the concentration and/or optional diafiltration step are operated in a manner favourable to the removal of trypsin inhibitors.

38. The process of claim 1 wherein a reducing agent is present during the extraction step to disrupt or rearrange the disulfide bonds of trypsin inhibitors to achieve a reduction in trypsin inhibitor activity.

39. The process of claim 21 wherein a reducing agent is present during the concentration and/or optional diafiltration step to disrupt or rearrange the disulfide bonds of trypsin inhibitors to achieve a reduction in trypsin inhibitor activity.

40. The process of claim 33 wherein a reducing agent is added to the concentrated and optionally diafiltered soy protein solution prior to drying and/or the dried soy protein product to disrupt or rearrange the disulfide bonds of trypsin inhibitors to achieve a reduction in trypsin inhibitor activity.

41. A soy protein product having reduced astringency.
